(19) United States
(12) Patent Application Publication
(10) Pub. No.: US 2009/0221979 A1
(43) Pub. Date: Sep. 3, 2009

(54) TOPSHEET FOR AN ABSORBENT ARTICLE

(76) Inventors: Chen-Cheng Huang, Taipei City (TW); Pao-Hao Huang, Taipei City (TW); Pao-Han Huang, Taipei City (TW)

Correspondence Address: SHOOK, HARDY & BACON LLP;INTELLECTUAL PROPERTY DEPARTMENT, 2555 GRAND BLVD, KANSAS CITY, MO, 64108-2613, US

(21) Appl. No.: 12/394,265

(22) Filed: Feb. 27, 2009

(30) Foreign Application Priority Data
Feb. 29, 2008 (TW) 097107029

(51) Int. Cl. A61F 13/511

(52) U.S. Cl. 604/367; 604/385.101

(57) ABSTRACT

A top sheet includes a sheet body having a flat liquid pervious base wall with first and second surfaces that are opposite to each other in a normal direction relative to the sheet body. The base wall has front and rear edges that are opposite to each other in a longitudinal direction. The sheet body further has a plurality of protrusions that protrude from the first surface of the base wall and that are distributed along the longitudinal direction as well as a widthwise direction which is transverse to the longitudinal direction. The protrusions are arranged in such a manner to form a plurality of open fluid channels thereamong. The fluid channels extend from the front edge to the rear edge of the base wall, and have dimensions in the normal and widthwise directions sufficient to provide capillary action.

### CROSS-REFERENCE TO RELATED APPLICATION

This application claims priority of Taiwanese Application No. 097107029, filed on Feb. 29, 2008.

### BACKGROUND OF THE INVENTION

1. Field of the Invention

This invention relates to a topsheet for an absorbent article, more particularly to a topsheet including a base wall and a plurality of strip-like protrusions protruding from the base wall to form a plurality of fluid channels for spreading liquid in a desired direction.

2. Description of the Related Art

U.S. Pat. No. 6,461,339 discloses an absorbent article that includes a liquid pervious topsheet, a liquid impervious backsheet, an absorbent core disposed between the topsheet and the backsheet, and a non-woven inner layer disposed between the topsheet and the absorbent core. The topsheet has an outer layer of a non-woven fabric, and a perforated plastic film layer that is in contact with the non-woven inner layer. The outer layer may be embossed, and serves mainly for providing softness and to absorb moisture so that a sticky or sweaty feel is not experienced by the user.

The outer layer is formed with an opening extending from a front edge of the absorbent article to a rear edge of the absorbent article for passage of a liquid to be deposited directly onto the perforated plastic film layer so that the liquid can rapidly pass into the absorbent core through the perforated plastic film layer and the non-woven inner layer.

The entire disclosure of U.S. Pat. No. 6,461,339 is incorporated herein by reference.

By virtue of the structures of apertures formed in the perforated plastic film layer, pores formed in the outer layer, and pores formed in the non-woven inner layer, liquid deposited on the outer layer can effectively pass through the apertures and the pores in a normal direction relative to the absorbent article and into the absorbent core, but cannot effectively flow in a lengthwise direction of the absorbent article. As a consequence, liquid flow from the outer layer into the absorbent core occurs only over a limited area of the absorbent article, i.e., a wet area where the liquid is deposited.

It has been proposed that the fibers used for forming the non-woven inner layer be oriented in a specific direction before they are entangled together so as to permit spreading of the liquid in the lengthwise direction and so as to increase the wet area over which the liquid flows through the outer layer, the perforated plastic film layer, and the non-woven inner layer and into the absorbent core. However, the non-woven inner layer thus formed is expensive.

Hence, there is a need for a topsheet that is cost effective and that can effectively cause spreading of liquid in the lengthwise direction.

### SUMMARY OF THE INVENTION

Therefore, an object of the present invention is to provide a topsheet having a configuration that can effectively cause spreading of a liquid body and dispense with the aforementioned non-woven inner layer employed in the prior art.

According to the present invention, a topsheet for an absorbent article comprises a flexible sheet body having a flat liquid pervious base wall with first and second surfaces that are opposite to each other in a normal direction relative to the sheet body. The base wall has front and rear edges that are opposite to each other in a longitudinal direction. The sheet body further has a plurality of strip-like protrusions that are spaced apart from each other, that protrude from the first surface of the base wall, and that are distributed along the longitudinal direction as well as a widthwise direction which is transverse to the longitudinal direction. Each of the protrusions has front and rear ends that face toward the front and rear edges of the base wall, respectively, and extends lengthwise from the rear end to the front end. The protrusions are arranged in such a manner to form a plurality of strip-like open fluid channels thereamong. The fluid channels extend from the front edge to the rear edge of the base wall, and have dimensions in the normal and widthwise directions sufficient to provide capillary action, thereby promoting spreading of a liquid body on the surface of the base wall.

### BRIEF DESCRIPTION OF THE DRAWINGS

In drawings which illustrate embodiments of the invention,

FIG. 1 is a fragmentary perspective view of the first preferred embodiment of a topsheet according to this invention;

FIG. 2 is a fragmentary sectional view of the first preferred embodiment;

FIG. 3 is a fragmentary perspective view of the second preferred embodiment of a topsheet according to this invention;

FIG. 4 is a fragmentary sectional view of the second preferred embodiment;

FIG. 5 is a fragmentary perspective view of the third preferred embodiment of a topsheet according to this invention;

FIG. 6 is a fragmentary sectional view of the third preferred embodiment;

FIG. 7 is a fragmentary perspective view of the fourth preferred embodiment of a topsheet according to this invention;

FIG. 8 is a fragmentary sectional view of the fourth preferred embodiment; and

FIG. 9 is a fragmentary sectional view of the fifth preferred embodiment of a topsheet according to this invention.

### DETAILED DESCRIPTION OF THE PREFERRED EMBODIMENTS

Before the present invention is described in greater detail with reference to the accompanying preferred embodiments, it should be noted herein that like elements are denoted by the same reference numerals throughout the disclosure.

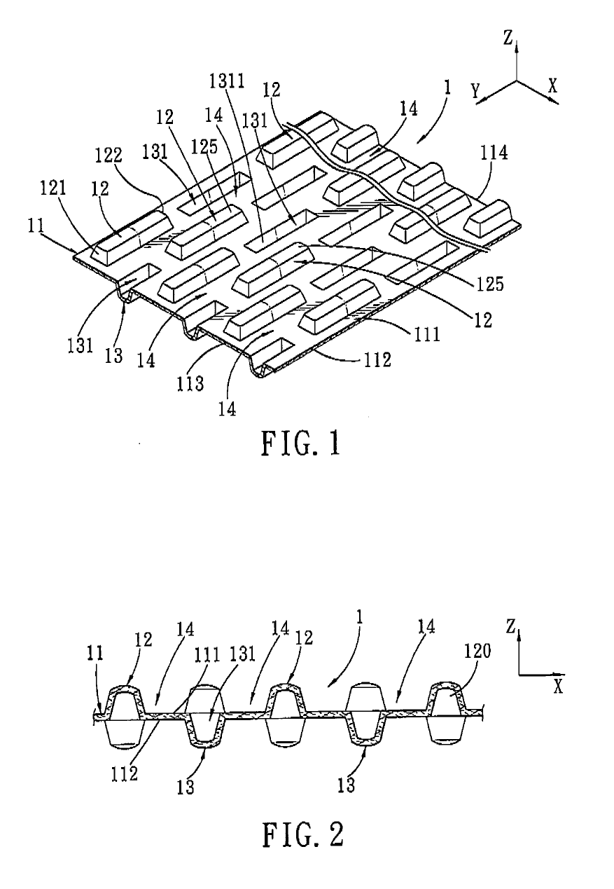

FIGS. 1 and 2 illustrate the first preferred embodiment of a topsheet for an absorbent article, such as a sanitary napkin, an incontinence pad, a diaper, and the like, according to this invention. The topsheet includes a flexible sheet body 1 having a flat liquid pervious base wall 11 with first and second surfaces 111, 112 that are opposite to each other in a normal direction (Z) relative to the sheet body 1. The base wall 11 has front and rear edges 113, 114 that are opposite to each other in a longitudinal direction (Y) that is substantially perpendicular to the normal direction (Z).

The sheet body 1 further has a plurality of strip-like first protrusions 12 that are spaced apart from each other. The first protrusions 12 protrude from the first surface 111 of the base wall 11 in the normal direction (Z), and are distributed along the longitudinal direction (Y), as well as along a widthwise direction (X) which is transverse to the longitudinal direction (Y).

In this embodiment, the sheet body 1 is made from a non-woven fabric that is formed with a plurality of open pores for passage of a liquid (not shown) therethrough in the normal direction (Z). It is noted that formation of the non-woven fabric can be conducted in a conventional manner, such as by entangling fibers together using simple mechanical techniques that do not involve orienting fibers in a specific direction.

Each of the first protrusions 12 has front and rear ends 121, 122 that face toward the front and rear edges 113, 114 of the base wall 11, respectively, and extends lengthwise from the rear end 121 to the front end 122. The first protrusions 12 are arranged in such a manner to form a plurality of strip-like open fluid channels 14 thereamong. The fluid channels 14 extend from the front edge 113 to the rear edge 114 of the base wall 11, and have dimensions in the normal and widthwise directions (Z, X) sufficient to provide capillary action, thereby promoting spreading of a liquid body (not shown) on the first surface 111 of the base wall 11 in the longitudinal direction (Y).

In this embodiment, each of the first protrusions 12 is hollow and defines a recess that opens at the second surface 112 of the base wall 11.

The sheet body 1 further has a plurality of strip-like second protrusions 13 that protrude from the second surface 112 of the base wall 11, and that are distributed along the longitudinal direction (Y), as well as along the widthwise direction (X). Each of the second protrusions 13 is hollow, and defines a strip-like trough 131 that opens at the first surface Ill of the base wall 11 and that extends lengthwise in the longitudinal direction (Y). The troughs 131 defined by the second protrusions 13 are alternately disposed with the first protrusions 12. Each of the troughs 131 is partially aligned in the widthwise direction (X) with at least one of the first protrusions 12. That is, each of the troughs 131 has a section 131 that is aligned in the widthwise direction (X) with a portion 125 of said at least one of the first protrusions 12. The troughs 131 have dimensions in the normal and widthwise directions (Z, X) sufficient to provide capillary action, thereby enhancing spreading of the liquid body in the longitudinal direction (Y).

In this embodiment, each of the first protrusions 12 extends lengthwise in the longitudinal direction (Y) so that each of the fluid channels 14 also extends in the longitudinal direction (Y). In addition, each of the fluid channels 14 is in fluid communication with an adjacent one of the fluid channels 14 so as to promote spreading of the liquid body also in the widthwise direction (X).

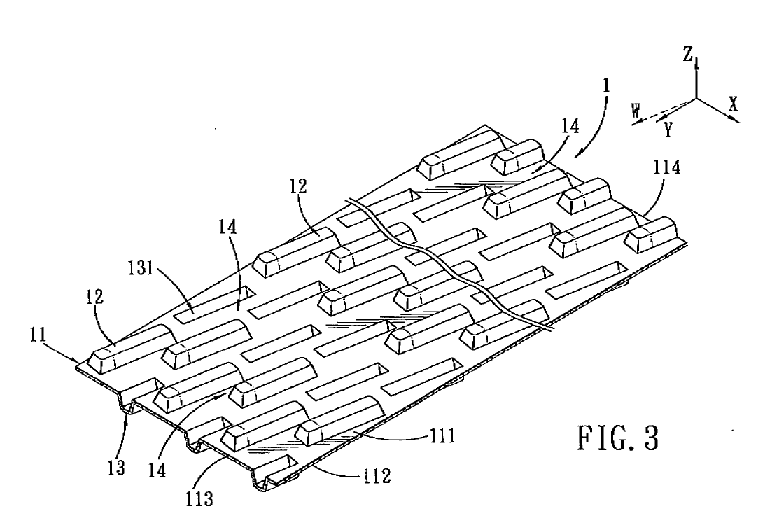

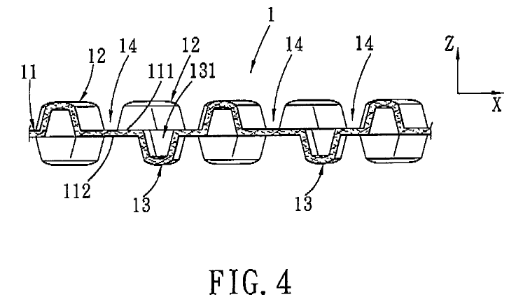

FIGS. 3 and 4 illustrate the second preferred embodiment of the topsheet according to this invention. The second preferred embodiment differs from the previous embodiment in that each of the first protrusions 12 extends lengthwise in a direction (W) that is slightly inclined relative to the longitudinal direction (Y). In some embodiments, each of the second protrusions 13 also extends lengthwise in the direction (W).

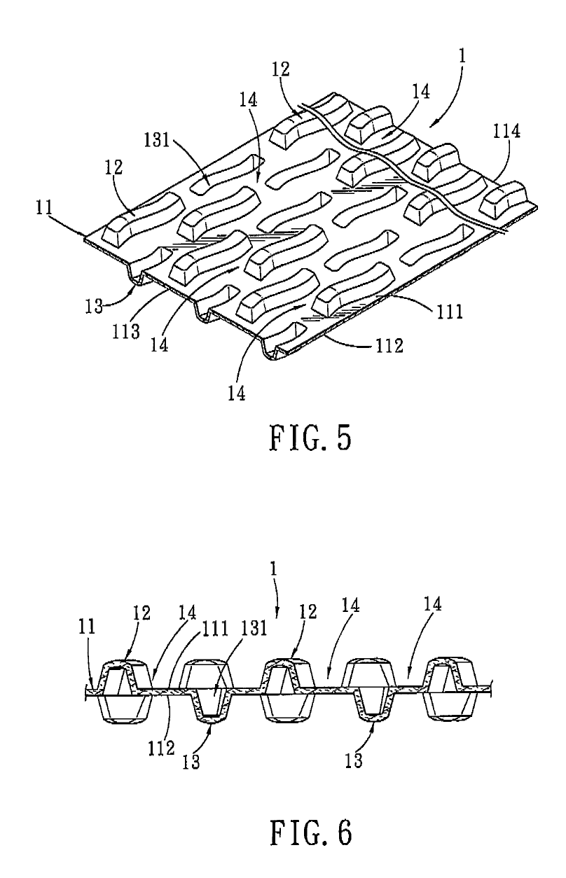

FIGS. 5 and 6 illustrate the third preferred embodiment of the topsheet according to this invention. The third preferred embodiment differs from the first embodiment in that each of the first protrusions 12 is curvy in shape. That is, in the third preferred embodiments, each of the first protrusions 12 extends lengthwise in a slightly meandering manner. In some embodiments, each of the second protrusions 13 is also curvy in shape.

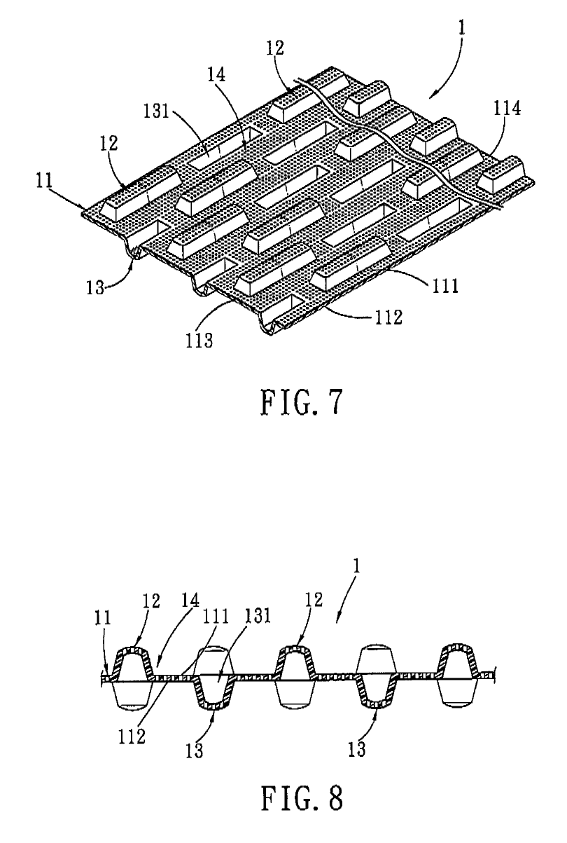

FIGS. 7 and 8 illustrate the fourth preferred embodiment of the topsheet according to this invention The fourth preferred embodiment differs from the first embodiment in that the sheet body 1 is made from a perforated plastic film which is formed with apertures for passage of liquid therethrough in the normal direction (Z). It is noted that in other preferred embodiments, the apertures can be formed only in the base wall 11 of the sheet body 1, and not in the first and second protrusions 12, 13 so that the first and second protrusions 12, 13 are liquid impervious.

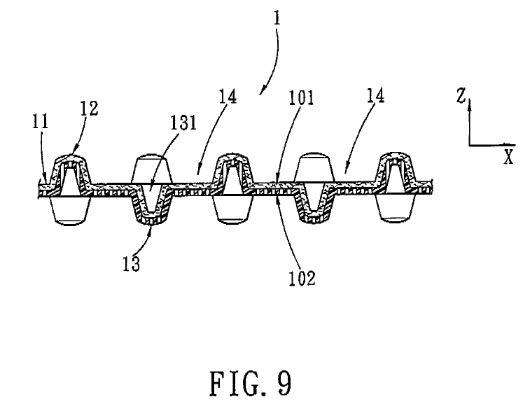

FIG. 9 illustrates the fifth preferred embodiment of the topsheet according to this invention. The fifth preferred embodiment differs from the first embodiment in that the sheet body 1 is made from a composite material having a bi-layer structure that has a non-woven layer 101 and a perforated plastic film layer 102 bonded to the non-woven layer 101.

With the inclusion of the first protrusions 12 and the troughs 131 in the sheet body 1 of the topsheet of this invention, a liquid body on the first surface 111 of the base wall 11 can rapidly spread in the longitudinal direction (Y), thereby considerably increasing the wet area of the base wall 11 over which liquid passes through both the base wall 11 as well as through walls that define the troughs 131. Ultimately, such a configuration of the present invention promotes transport of liquid into the absorbent article.

With the invention thus explained, it is apparent that various modifications and variations can be made without departing from the spirit of the present invention. It is therefore intended that the invention be limited only as recited in the appended claims.

What is claimed is:

1. A topsheet for an absorbent article, comprising:
a flexible liquid pervious sheet body having a flat liquid pervious base wall with first and second surfaces that are opposite to each other in a normal direction relative to said sheet body, said base wall having front and rear edges that are opposite to each other in a longitudinal direction, said sheet body further having a plurality of strip-like first protrusions that are spaced apart from each other, that protrude from said first surface of said base wall, and that are distributed along the longitudinal direction as well as a widthwise direction which is transverse to the longitudinal direction, each of said first protrusions having front and rear ends that face toward said front and rear edges of said base wall, respectively, and extending lengthwise from said rear end to said front end;
wherein said first protrusions are arranged in such a manner to form a plurality of strip-like open fluid channels thereamong, said fluid channels extending from said front edge to said rear edge of said base wall and having dimensions in the normal and widthwise directions sufficient to provide capillary action, thereby promoting spreading of a liquid body on said first surface of said base wall.

2. The topsheet of claim 1, wherein each of said first protrusions is hollow.

3. The topsheet of claim 1, wherein said sheet body further has a plurality of strip-like second protrusions that protrude from said second surface of said base wall, and that are distributed along the longitudinal direction as well as the widthwise direction, each of said second protrusions being hollow and defining a strip-like trough that opens at said first surface of said base wall, said troughs defined by said second protrusions being alternately disposed with said first protrusions.

4. The topsheet of claim 3, wherein each of said troughs is partially aligned in the widthwise direction with at least one of said first protrusions.

5. The topsheet of claim 1, wherein each of said first protrusions extends lengthwise in the longitudinal direction so that each of said fluid channels extends in the longitudinal direction.

6. The topsheet of claim 1, wherein each of said first protrusions extends lengthwise in a direction slightly inclined relative to the longitudinal direction.

7. The topsheet of claim 1, wherein each of said first protrusions is curvy in shape.

8. The topsheet of claim 1, wherein said sheet body is made from a non-woven fabric.

9. The topsheet of claim 1, wherein said sheet body is made from a perforated plastic film.

10. The topsheet of claim 1, wherein said sheet body is made from a composite material having a bi-layer structure that has a non-woven layer and a perforated plastic film layer bonded to said non-woven layer.
